(19) United States
(12) Patent Application Publication
(10) Pub. No.: US 2014/0276001 A1
(43) Pub. Date: Sep. 18, 2014

(54) Device and Method for Image-Guided Surgery

(71) Applicant: Queen's University at Kingston, Kingston (CA)

(72) Inventors: Tamas Ungi, Kingston (CA); Andras Lasso, Kingston (CA); Gabor Fichtinger, Kingston (CA)

(73) Assignee: Queen's University at Kingston, Kingston (CA)

(21) Appl. No.: 14/209,232

(22) Filed: Mar. 13, 2014

Related U.S. Application Data
(60) Provisional application No. 61/791,742, filed on Mar. 15, 2013.

(51) Int. Cl. A61B 8/08; A61B 8/14; A61B 8/00; A61B 5/06

(52) U.S. Cl. CPC A61B 8/0841; A61B 5/064; A61B 8/14; A61B 8/461
USPC 600/424

(57) ABSTRACT

In one aspect the invention provides a reference device that enhances image-guided surgical interventions. The reference device is tracked by the imaging system and used to verify the accuracy of the intervention tool placement before and during the intervention. The reference device holds a reference sensor in a position aligned with patient anatomy, so that images are displayed in the correct orientation to the operator, aiding in target recognition and better navigation. Also provided are methods using the reference device and programmed computer media for implementing at least a part of the methods.

### RELATED APPLICATION

This application claims the benefit of the filing date of U.S. Patent Application No. 61/791,742, filed on 15 Mar. 2013, the contents of which are incorporated herein by reference in their entirety.

### FIELD

This invention relates generally to image-guided surgical interventions. More specifically, the invention relates to ultrasound guidance of surgical interventions and a tracked reference device therefor.

### BACKGROUND

A significant drawback to use of ultrasound images in guiding medical interventions is the general difficulty in recognizing target structures in the images. Moreover, the simultaneous manipulation of the ultrasound transducer and the interventional tool (e.g., a needle) requires considerable skill and experience.

Some interventions (e.g., spinal) are performed under X-ray fluoroscopic or computed tomography (CT) guidance, because the interpretation of X-ray based images is not hampered by muscle and ligament layers between the skin and the target. CT and X-ray-based imaging modalities visualize the target anatomy and the needle much better that ultrasound does, but they involve significantly larger and more expensive equipment than ultrasound, and they introduce ionizing radiation to the patient and to a larger extent to the operator who performs these procedures on a regular basis.

Using electromagnetically tracked ultrasound transducers and interventional tools to enhance ultrasound guided interventions with computer navigation has made some procedures accessible for less experienced physicians. Nevertheless, applying electromagnetic tracking in certain procedures, such as spinal interventions, has been hampered because of the difficulty in interpreting spine anatomy in ultrasound images, and in locating relatively small and deep targets under the skin surface. Electromagnetic tracking also suffers from poor accuracy and interference with metal parts in the vicinity of the operating space.

### SUMMARY

Provided herein is a reference device for surgery, comprising: a base portion, including; a socket that accepts a tracking sensor in a pre-defined orientation; one or more reference divots that accept at least a portion of a surgical intervention tool, the one or more reference divots being substantially transparent to one or more imaging modalities; and a plurality of anatomical direction markers that provide alignment of the reference device with the patient's anatomy.

In one embodiment, the base portion interfaces with a patient's anatomy substantially non-invasively. In another embodiment, the base portion interfaces with an object fixed to the patient's anatomy. In another embodiment, the base portion interfaces with a surface in proximity to a surgical invention site.

In one embodiment, the socket accepts an electromagnetic tracking sensor that is used as a reference point in tracking at least one of position, orientation, and trajectory of the surgical intervention tool in three-dimensional space. In these embodiments, locations of the one or more reference divots are known with respect to the orientation of the tracking sensor.

Also provided is method of medical imaging; comprising: disposing a reference device in a selected orientation with respect to an intervention space of a subject, the reference device providing anatomical orientation of tracked medical images within the intervention space; using an ultrasound imaging system to obtain tracked medical images of the intervention space; and using the anatomical orientation provided by the reference device to display the tracked medical images in the intervention space in a perspective that corresponds to an operator's perspective.

The method may further comprise displaying one or more of position, orientation, and trajectory of a tracked intervention tool with respect to the tracked medical images in the intervention space. The method may further comprise verifying at least one of position, orientation, and trajectory of the tracked intervention tool with respect to the tracked medical images in the intervention space, by placing the tracked intervention tool at one or more locations on the reference device, wherein the locations are known with respect to the position of a sensor associated with the reference device.

In one embodiment, verifying further comprises providing an indication to the system when the tracked intervention tool is disposed at each of the one or more locations.

The method may further comprise disposing an electromagnetic sensor in a known position and orientation with respect to the reference device. The method may further comprise aligning a tracked medical image with a volumetric medical image. The method may further comprise displaying the tracked medical images substantially in real time.

In one embodiment, the medical imaging system is an ultrasound imaging system or a tomographic imaging system. In one embodiment, the tracked medical image is an ultrasound image.

Also provided is programmed media for use with a computer, comprising: a computer program stored on non-transitory storage media compatible with the computer, the computer program containing instructions to direct the computer to perform the following steps: obtain tracked medical images of an intervention space from a medical imaging system; and use anatomical orientation provided by a tracked reference device to display the tracked medical images in the intervention space in a perspective that corresponds to an operator's perspective.

### BRIEF DESCRIPTION OF THE DRAWINGS

For a greater understanding of the invention and to show more clearly how it may be carried into effect, embodiments are described below, by way of example, with reference to the accompanying drawings, wherein:

FIG. 1 is a perspective view of a reference device according to one embodiment;

FIG. 2 is a schematic diagram of a typical tracked ultrasound-guided needle navigation system showing a tracked reference device integrated into the system;

FIG. 3 is a schematic representation of the coordinate systems and transforms in a tracked ultrasound-guided needle navigation system according to an embodiment described herein;

FIG. 4 is a perspective view of the reference device of FIG. 1 showing known divot positions (P₁₋₄) and tip positions (P′₁₋₄) of a tracked needle when the needle tip is placed in the divots;

FIG. 5 is a flowchart showing an example of a workflow of intervention tool (e.g., a needle) insertions using a reference device as described herein;

FIG. 6 is a flowchart shown the surgical workflow for ultrasound-based registration in Example 1;

FIG. 7 shows planning of pedicle screw locations using landmark points (dots) on the CT image and the screw plan;

FIG. 8 shows planned pedicle screw locations for a healthy spine model (A and C) and a degenerative spine model (B and D); posterior views are shown in the top row (A and B) and right oblique view with semi-transparent bone models in the bottom row (C and D);

FIG. 9 shows four selected landmarks for vertebra registration (left panel) and US snapshots (right panel) to illustrate how to guide the sagittal plane to the facet joint area; the semi-transparent vertebra overlaid on US snapshots is only for illustration, and is not visible during actual landmark definition;

FIG. 10 shows an overview of pedicle screw plan positions as defined in the CT image (grey rods) and as registered using US snapshots (black rods) in a healthy spine model (A) and a degenerative spine model (B);

FIG. 11 is a scatter plot of translation errors of individual TUSS-based screw positions relative to CT-based screw positions in the left-right, inferior-superior anatomical plane, for healthy and degenerative spine models;

FIG. 12 is shows the dual 3D navigation layout of a graphical user interface used in a spinal needle insertion work phase;

FIG. 13 shows a bull's-eye view orientation for intuitive navigation used in spinal needle insertion, wherein letters denote directions in the patient or phantom coordinate system: S, superior; 1, inferior; P, posterior; A, anterior; R, right; and L, left;

FIG. 14 is flowchart showing workflow steps for the needle insertion experiments;

FIG. 15 shows registered bone surface model images with tracked needle positions used for verification of spinal needle insertion outcomes: needle position in a synthetic human spine model using a bone surface model from a registered CT volume (left panels); corresponding orthogonal fluoroscopic images (right panels) were used as an independent verification method for needle tip position; arrows point at the needle tips;

FIG. 16 is a spinal needle navigation scene in a 3D Slicer with dual 3D view showing multiple facet joint targets in a cadaveric lamb model; the tracked needle (visualized as a black stick) is placed in target “P1” (upper panels); registration of the CT volume to the EM tracker results in a scene augmented with the bone surface model, used for training and validation (bottom panels); and

FIG. 17 shows plots of targeting error and insertion time of all needle insertions in the a system accuracy study; upper panel: scatter plot of needle tip targeting error vs. insertion number; lower panel: scatter plot of insertion time vs. insertion number.

### DETAILED DESCRIPTION OF EMBODIMENTS

Embodiments described herein provide rapid (e.g., substantially instantaneous or real-time) tracking at the intervention site of an invention tool, thereby improving the accuracy of surgical interventions and helping physicians avoid adverse events.

One aspect of the invention provides a hardware reference device that enhances image-guided interventions. The reference device is tracked by the system and used to verify the accuracy of the intervention tool (i.e., a surgical tool) placement before and during the intervention. The reference device holds a reference sensor (e.g., electromagnetic (EM) sensor in a position aligned with patient anatomy. This is used to show the ultrasound images in the correct orientation to the operator, aiding in target recognition and better navigation.

An embodiment of the tracked reference device is shown in FIG. 1. The device 12 may be constructed as one piece or substantially one piece, made of a suitable material such as plastic. Embodiments constructed as such are low cost and may be for single use and disposable. Alternatively, the device may be re-usable and accordingly made of a material that can withstand sterilization. The device has a base portion 30. The term “base portion” as used herein generally refers to a structure on or in which further features, such as those listed below, are disposed.

In one embodiment the base portion 30 may non-invasively interface with the patient's anatomy. The base portion 30 may have a surface that is generally shaped to fit on the exterior anatomy of the patient in the vicinity or region of the patient where the intervention is to take place. For example, the base portion 30 may have a curved surface, for use on a patient's skull. In the embodiment of FIG. 1, the base portion 30 has a substantially flat surface, with leaves 30a and 30b in the left and right directions, respectively, and is suitable for, e.g., interventions on a patient's back, such as spinal injection or placement of pedicle screws. Such an embodiment is easily and non-invasively affixed to the patient's skin near the intervention site using, e.g., tape. In other embodiments, the base portion may be adapted to attach to a patient's anatomy using a pin or other structure. Alternatively, the base portion may be adapted to removably engage a needle, pin, screw, or the like which has been fixed to the patient's anatomy. In particular, when a more rigid connection is needed between the device and the patient, the device may be fixed to a bone of the patient via a threaded pin or screw. In such an embodiment, the base portion comprises a mechanical interface that can be fixed to the pin or screw. Further, it will be appreciated that the device need not be attached or fixed to the patient. For example, in some procedures the device may be placed on a suitable surface next to the patient.

Features of the tracked reference device include one or more anatomical direction markers, a socket that accepts or accommodates a tracking sensor in a pre-defined orientation, and one or more reference divots that accept at least a portion of the intervention tool during verification. In general, these features are disposed in or on the base portion. The divots may be sized or shaped to accept a specific tool, such as, e.g., a needle. The divots may be sized or shaped to accept a specific position and/or orientation of a tool. In one embodiment the divots are transparent or substantially transparent to one or more imaging modalities such as ultrasound and tomography. The embodiment of FIG. 1 includes six anatomical direction markers corresponding to standard anatomical orientation: letters L (left), R (right), P (posterior), A (anterior), S (superior), I (inferior), a socket 32 that holds a reference tracking sensor in a pre-defined orientation, and four reference divots 34, numbered 1-4.

The tracked reference device may be used with an imaging system, an embodiment of which is shown in FIG. 2. In this example an EM signal is provided to the patient 2 by an EM transmitter 10 and the signal is tracked by an EM tracker 18. A computer 20 controls the ultrasound transducer 14. A tracked intervention tool having a sensor mounted thereon is shown at 16, and the tracked reference device at 12. Navigational software may be run on the ultrasound computer 20 or optionally on a separate computer 22. The system may be integrated into any existing or commercially available tracked ultrasound and tool systems, such as, for example, the Sonix Touch UPS system (Ultrasonix Medical Corporation, Richmond, B.C., Canada).

Accurate navigation of the intervention tool 16 ensures that the tool is close to a target when the virtual tool tip is at the target point on the navigation computer display. The system prevents loss of accuracy of the navigation and mitigates any risk of misplacement of the tool. The system may be configured to warn the operator in case of insufficient accuracy before the needle insertion.

In one embodiment, virtual camera alignment in the navigation display is achieved by a series of coordinate transforms, an embodiment of which is illustrated in FIG. 3. The reference device 12 creates a link between the reference sensor coordinate system and the navigation display coordinate system. This link is implemented using the anatomical direction marks on the reference device that are aligned with the patient anatomy when fixing the reference device near the intervention site. The reference tracking sensor is held in the socket 32 of the reference device 12 in a pre-defined position and orientation. Since all tracked positions are transformed to the coordinate system of the reference sensor, they are sent to the navigation system in a conventional anatomical coordinate system.

The navigation system uses the sensed positions in the reference sensor coordinate system to present virtual models of the ultrasound image, the intervention tool, and optionally additional patient images to serve tool navigation needs. Assessment of tool tracking accuracy before insertion into the patient is performed using the reference divots 34 on the reference device 12. Known (P) and tracked (P′) positions of the tool relative to the reference sensor are compared (FIG. 4). The method uses known ground truth positions of the divots 34 with respect to the reference sensor. The ground truth positions may be computed using the mechanical design of the device, and verified using high accuracy tracker equipment in a controlled manufacturing environment. The tracked tool tip is placed in each divot before insertion into the patient, and the operator sends an indication to the system when the tool is placed in each divot. For example, the indication may comprise pivoting the tool in the divot or engaging a switch, etc. If a large discrepancy is detected between tracked and ground truth tool tip positions, a warning may be sent to the operator that the tool tracking is not reliable. An example of a workflow is shown in the flowchart of FIG. 5.

The maximum acceptable difference between known and tracked tool tip positions depends on the size of the target. For example, typical needle targets in the spine require an accuracy of 1-3 mm.

Another aspect of the invention comprises a method that enhances ultrasound-guided interventions. The method works with an ultrasound scanner and a surgical intervention tool, both electromagnetically tracked in 3-dimensional space in real-time. The method may be used in conjunction with the tracked reference device described herein to perform verification before and during the surgical procedure. The method may also create a 3-dimensional augmented reality computer scene with the ultrasound image and the 3-dimensional model of the intervention tool. A feature of the method is that the tracked medical images in the intervention space are displayed in a perspective that corresponds to an operator's perspective.

At least a portion of the method may be implemented in software, including, for example, an algorithm, and stored on non-volatile computer storage media, and run on a suitable computer. The computer may be part of an imaging system. In one embodiment, the imaging system is part of a tracked ultrasound-guided intervention tool navigation system.

As described herein, a target (i.e., an intervention site) is identified in the computer guidance scene, and therefore the intervention tool can be introduced to the target using the computer scene, rather than via direct, live ultrasound imaging. This focuses the attention of the operator to the tool insertion, and ensures higher accuracy even at an early stage of the operator learning curve.

When a pre-operative tomographic image is available for the patient, the reference device allows alignment of the tomographic image with the ultrasound tracking coordinate system, which results in fusion of tomographic and ultrasound images. The tracked reference device ensures correct orientation of the ultrasound image; therefore the dimensionality of the alignment space is reduced to four degrees of freedom (translation+rotation around the left-right axis) from the original six degrees of freedom (including two other rotation axes). 3-D translation alignment with one rotation can be performed robustly and quickly. In such a way, fused ultrasound-tomography images may be made available for insertion planning in a routine procedure.

The invention is further described by way of the following non-limiting examples.

### Example 1

### Tracked Ultrasound Snapshots in Percutaneous Pedicle Screw Placement Navigation

### Introduction

Pedicle screw placement is considered the standard of care in many spinal deformation diseases. Registration of a preoperative CT with an intraoperative stereotactic guidance system can completely eliminate ionizing radiation during pedicle screw placement, while the accuracy and success of pedicle screw placement remains excellent. This registration method requires landmark localization in both the CT and the intraoperative tracking coordinate systems. These landmarks determine the transformation that fuses the preoperative CT with the intraoperative virtual reality navigation scene. In this study, a tracked ultrasound snapshot (TUSS) technique was used with a tracked reference device to find these landmarks through non-invasive ultrasound (US) imaging. The tracked reference device may be a device as described above and shown in FIG. 1. The resulting registration transformation was used to place the pedicle screw plans in the surgical navigation coordinate system.

Automatic CT to US image registration methods are promising alternatives to manual landmarking of US images. However, a method to compute a reliable registration transform on all reported experimental test cases at a satisfactory accuracy is not known. Since intraoperative conditions could further reduce the success rate of automatic methods, manually defined landmarks were considered the most accurate available CT registration method for this procedure.

Pedicle screw positions were planned using a preoperative CT scan. The plans were later registered to the surgical navigation coordinate system using TUSS landmarks. The registration was evaluated based on clinical safety parameters of the registered pedicle screw plans in two patient-based phantom models.

### Materials and Methods

The surgical workflow is shown in FIG. 6. A preoperative CT scan was used to define pedicle screw positions. Registration landmarks were defined on the CT scans of vertebrae. In the intraoperative phase, corresponding landmarks were localized using TUSS. After landmark registration, the CT-based pedicle screw plans were transformed to the intraoperative navigation coordinate system for evaluation. Landmark-based registration transformation was computed using Horn's closed form solution (Horn, B.K.P., “Closed-form solution of absolute orientation using unit quatemions”, Journal of the Optical Society of America A, Vol. 4:629-642, 1987).

The intraoperative navigation system was as shown in FIG. 2, except a spine phantom ras used instead of a patient. The system included a Sonix Tablet (Ultrasonix, Richmond, BC, Canada) US machine 20, with integrated GPS extension for electromagnetic position tracking. This tracker hardware extension included a DriveBay electromagnetic tracker (Ascension Technology Corporation, Milton, Vt., USA) and an adjustable arm that held the EM transmitter. Alternatively, a tracked reference device as described herein (e.g., as in FIG. 1) could be used The tracked intervention tool 16 was a Jamshidi needle, and the tracked reference device 12 was fixed to the phantom. The 3-D navigation software was implemented as an extension (SlicerIGT) for the 3D Slicer application. The navigation software ran on a dedicated computer 22, getting real time tracking and US image data through network connection from the US machine, using the OpenIGTLink data communication protocol (Tokuda, J., et al., “OpenIGTLink: an open network protocol for image-guided therapy environment”, Int. J. Med. Robot. 5, No. 4 (December 2009):423-434).

The registration workflow was carried out in two patient-based lumbar spine models. One model was based on healthy anatomy and the other on degenerative spine disease. The tests involved L2-L5 segments in each spine model, with two pedicle screw plans in each vertebra.

Two rapid prototyped spine segments of L2-L5 were used for the evaluation of the TUSS-based pedicle screw plan registration. The spine models were generated by manually contouring healthy and degenerative spine CT scans. Planning of the pedicle screws was done using four points in the CT image of each pedicle (FIG. 7). Optimal positions and orientations of the screws were determined by manually placing these points on the left and right edge of the pedicles on coronal CT slices in an anterior and a posterior section of the pedicles. Corresponding predefined points on the screw models were registered to these CT points to obtain optimal positions of the screws for each pedicle. FIG. 8 shows planned screw positions for the healthy spine model (A and C views) and the degenerative spine model (B and D views). Posterior views are shown in the top row (A and B) and right oblique view with semi-transparent bone models in the bottom row (C and D). All planned screws were 4 mm in diameter and 50 mm in length.

Registration from the CT image to the surgical navigation scene was done using anatomical landmark points on vertebrae. For this, landmarks (e.g., articular processes of vertebrae) were identified that were visible in both CT and intraoperative US images.

Lumbar spine images of 10 human subjects were examined to verify visibility of anatomical landmarks on US images. The study protocol was approved by the Health Sciences Research Ethics Board at Queen's University. Written informed consent was obtained from subjects prior to participation in the study. The clinical parameters of the examined population are shown in Table 1. Registration landmarks were defined as the most posterior points of the four articular processes of each vertebra.

TABLE 1
Clinical parameters of human subjects.

| Parameter | Value |
|---|---|
| Height (m) ± SD | 171.2 ± 8.1 |
| Weight (kg) ± SD | 75.9 ± 20.0 |
| Body mass index (BMI) ± SD | 25.7 ± 6.2 |
| Age (years) ± SD | 29.1 ± 8.2 |
| Sex (male/female) | 5/5 |

Finding the articular processes with US imaging can be a difficult task. Therefore, an axial tracked US snapshot was taken to help find the intersecting sagittal US planes that correspond to the facet joint regions, as shown in FIG. 9. US landmark points were defined on sagittal tracked US snapshots. FIG. 9 shows four selected landmarks for vertebra registration (left panel). US snapshots (right panel) illustrate how to guide the sagittal plane to the facet joint area. The semi-transparent vertebra overlaid on US snapshots is only for illustration, and is not visible during actual landmark definition.

### Results and Discussion

The selected four registration landmarks were visible in all 10 human subjects, and in all patient-based simulation phantoms. All vertebrae in the two phantom models were successfully registered using US landmark points. FIG. 10 shows an overview of positions of the US-based pedicle screw plans (in black) compared to the ground truth positions of the plans (in grey), along with semi-transparent vertebrae in the healthy (A) and degenerative (B) models. Position and orientation differences between CT-based and US-based pedicle screw plans are summarized in Table 2 for all anatomical directions and axes.

Translational errors were measured at the center of the screw plan, which was positioned near the center of the pedicles during the planning phase. Orientation errors were decomposed into three Euler angles using the left-right, posterior-anterior, and inferior-superior anatomical axes.

Translational error in the coronal plane of individual screw centers was plotted (FIG. 11), because this projection of the error data is most relevant from a clinical complications perspective. The maximum translation error (3.51 mm) occurred in the superior direction in the degenerative model. Perforation of the pedicle wall by the TUSS-based screw plans was not detected in any of the pedicles.

TABLE 2
Translation (position) and orientation error of the US-based pedicle screw
center relative to the CT-based pedicle screw center. R: right, A: anterior,
S: superior directions. L-R: left-right, P-A: posterior-anterior, I-S:
inferior-superior rotation axes. SD: standard deviation.

| | Healthy Model | Degenerative Model |
|---|---|---|
| | Mean ± SD | Mean ± SD |
| Translation R (mm) | 0.16 ± 0.19 | 0.55 ± 0.59 |
| Translation A (mm) | −0.01 ± 1.22 | −0.35 ± 0.40 |
| Translation S (mm) | 0.68 ± 0.38 | 1.28 ± 1.37 |
| Rotation L-R (deg) | 1.92 ± 1.95 | 1.60 ± 1.56 |
| Rotation P-A (deg) | −0.05 ± 0.42 | 0.81 ± 1.15 |
| Rotation I-S (deg) | 0.40 ± 0.99 | −0.79 ± 0.46 |

The results confirm that TUSS is a useful tool in pedicle screw navigation, potentially improving the safety and reducing ionizing radiation in spinal fusion surgeries. Landmarks on TUSS images provide sufficient information to register the preoperative screw plans with the surgical navigation system. The translational errors were not uniform in all directions, and the deviation of positions was largest in the inferior-superior anatomical direction. This may be attributed to the elongated shape of the facet joints in the same direction, because facet joints were used as landmarks for US-CT registration. However, the errors were minor and would not detrimentally affect the intervention outcome in a patient. Moreover, the method avoids or substantially reduces the requirement for X-ray, thereby reducing radiation burden on operators and costs.

### Example 2

### Spinal Needle Navigation

This example provides a spinal needle insertion navigation system using tracked US snapshots (TUSS) that allows US-guided needle insertions without holding the US probe at the insertion site. The TUSS navigation software platform enables rapid development of image-guided needle placement applications, as well as other interventions, using tracked US for various anatomical targets and clinical indications. TUSS navigation was tested by five orthopedic surgeon residents in this study, guiding facet joint injections in cadaveric lamb and synthetic human spine models. Also reported is the targeting accuracy of the navigation system and a comparison with freehand US-guided needle placement.

### Materials and Methods

The navigation system consisted of a data acquisition and a visualization component. These components used network communication, and were run on two separate computers: the US machine collected image and tracking data, and the navigation computer was responsible for visualization. The system is as shown in FIG. 2.

images were acquired using a SonixTouch (Ultrasonix, Richmond, BC, Canada) US machine with a GPS extension. The GPS extension used the DriveBay EM position tracker (Ascension Technology Corporation, Milton, Vt., USA) with an adjustable arm to conveniently hold the EM transmitter close to the target area. An L14-5GPS linear array US transducer (Ultrasonix) and a 19-gauge nerve block needle (Ultrasonix) were tracked using built-in pose sensors. An additional Model 800 EM tracking sensor (Ascension Technology Corporation) attached to the target phantom or specimen served as the coordinate reference. Alternatively, a tracked reference device as described above with respect to FIG. 1 may be attached to the target phantom or specimen. A gigabit Ethernet network connected the US machine to the navigation computer. The navigation computer had an Intel Core2Quad processor, 3 GBRAM, NVIDIA GeForce 8800 GT graphics card, and ran under Windows XP operating system.

The software components of the navigation system included the PLUS (Public Library for Ultrasound) open-source software package to operate the US machine and the electromagnetic tracker. PLUS provides an abstraction layer for specific hardware programming interfaces and, importantly, it synchronizes the image and tracker data streams. The OpenIGTLink broadcaster application of the PLUS package was used to send the tracked US image frames to the navigation computer through the OpenIGTLink communication protocol (Tokuda, J., et al. “OpenIGTLink: an open network protocol for image-guided therapy environment”, Int. I. Med. Robot. 5, No. 4 (December 2009):423-434).

The navigation computer received the tracked US images, and provided the graphical user interface for needle guidance. The navigation software was implemented as an interactive module for the 3D Slicer application framework. This module, named LiveUltrasound, is shared under the open-source license of 3D Slicer1. It provides real-time visualization of the tracked US images and the tracked needle in the three-dimensional graphical views of 3D Slicer, as well as the ability to take tracked US snapshots for TUSS guidance

The navigation software provided needle guidance along an insertion plan. The plan was defined in 3D Slicer by the entry point and target point, i.e., the planned location of the needle piercing the skin and the planned final needle tip position, relative to the tracked US image. The dual 3-D view layout with an insertion plan is shown in FIG. 12. One of the 3-D views was set to “bull's-eye view”, in which the virtual camera superimposed the target and entry points. Coincidence of the target and entry points indicated correct virtual camera orientation. The other 3-D view was set to “progress view”, showing the US image plane parallel to the virtual camera image plane and was used to monitor the current penetration depth of the needle.

The orientations of the bull's-eye and progress views were aligned with the position of the operator, with respect to the patient (FIG. 13). The direction of needle motion towards the operator was shown in the bull's-eye view as a downward motion relative to the navigation monitor, while the progress view showed this motion as towards the camera. This arrangement provided intuitive hand-eye coordination during needle insertion.

A total of five orthopedic surgery residents participated in this study as operators to test the TUSS-guided needle navigation. None of the operators had used any form of tracked US needle guidance before performing the experiments. This study was approved by the Queen's University Health Sciences and Affiliated Teaching Hospitals Research Ethics Board.

Ultrasound-guided facet joint injection was not performed routinely by the operators, therefore, they had to learn how to identify the facet joint in the synthetic human spine and cadaveric lamb model. The phantom and the lamb cadavers were scanned using GE LightSpeed CT scanner (GE Healthcare, Chalfont St. Giles, UK), at an image resolution of 512×512 pixels and 0.625 mm slice distance. Bone surface models were extracted from the CT volumes using an intensity threshold. The surface model was registered and visualized together with the tracked US during the training. Surface markers on the synthetic human spine phantom, and nonferromagnetic metal screws in the cadaveric lamb models were used as landmarks for rigid registration between the C′I′ image and the EM position tracking system. During deliberate practice, the 3-D bone surface models were overlaid on the tracked US image in the navigation scene for the operators to learn the position of the facet joints in US with respect to the 3D anatomy. The training session did not involve handling of the tracked needle.

Each needle insertion procedure consisted of three main phases (FIG. 14). In the planning phase, the operator located the target by US, and one or more tracked snapshot US images were taken by the navigation software. Target and entry points were marked on the US snapshots. In the insertion phase, the navigation 3-D views were adjusted to the planned needle direction before they appeared to the operator on the navigation monitor in the dual 3-D view. Using the navigation scene, the operator aligned the tracked needle tip on the entry point, and then aligned the needle angle with the entry-target line of the insertion plan using the bull's-eye view. Finally, the operator inserted the needle along the planned trajectory, while observing the bull's-eye and progress views for real time feedback on the position of the needle relative to the insertion plan. The needle insertion was considered complete when the tip of the needle in both the bull's eye and progress views overlapped with the target point of the needle plan.

In the verification phase, two orthogonal X-ray images were acquired using a GE OEC 9800 fluoroscopy system (GE Healthcare, Chalfont St. Giles, UK) to assess the true needle tip position relative to the planned target. This phase is expected to be eliminated from the workflow, once sufficient evidence proves the reliability of TUSS guidance.

Tracked US snapshot navigation of needle insertion was studied in three experimental setups. Each experiment focused on different aspects of the navigation method. Table 3 summarizes major features of the experiment.

TABLE 3
Summary of Experimental Features

| Objective | Procedure | Endpoint |
|---|---|---|
| System | Target copper spheres in | Distance between target |
| accuracy | clear plastic gel | and needle tip |
| Human | Target facet joints in | Fluoroscopic verification |
| anatomy | synthetic human spine | |
| | models | |
| Biological | Target facet joints in fresh | Fluoroscopic verification, |
| tissue | cut lamb lumbar spine | procedure time |
| | regions. | |

First, targeting accuracy was studied using small artificial targets without anatomical landmarks. Copper spheres of 1.6 mm diameter were placed in acoustically clear Plastisol gel (M-F Manufacturing Company, Inc., Fort Worth Tex.). The needle tip was navigated to these targets using TUSS, and its distance from the surface of the copper spheres was measured using orthogonal X-ray projection images. Second, feasibility in human anatomy was tested using a synthetic, rapid prototyped spine model, placed in Plastisol gel. Cellulose (15 g/l) was mixed to the gel to simulate acoustic speckle of real soft tissue. The spine model was painted with X-ray contrast material (barium-sulphate) to show contrast on fluoroscopic images. The needle was navigated to the facet joints of this spine model using TUSS. Success or failure of needle placement was assessed using two X-ray projection images by a radiologist, blinded to the identity of operators. Registered bone surface model with tracked needle positions were also available during verification of insertion outcomes. For example, FIG. 15 shows needle position in the synthetic human spine model using the bone surface model from the registered CT volume (left panels). Corresponding orthogonal fluoroscopic images (right panels) were used as an independent verification method thr needle tip position. In FIG. 15, arrows point at the needle tips. This helped with the interpretation of needle positions relative to the bone anatomy.

Third, feasibility in biological tissue was tested using two fresh cut lamb lumbar spine regions. Tracked needles were navigated to the facet joints of the spine using TUSS. In order to assess the difference between TUSS-based navigation and freehand US-guided needle placement without position tracking, the cadaveric lamb model facet joint needle insertions were repeated in the same model without TUSS by all operators. Success of each insertion was assessed in the same way as in the synthetic human spine model. Needle insertions in the synthetic human spine phantom and the lamb model were carried out in groups to reduce experiment time. TUSS images were taken from the tracked live US stream for facet joints of five consecutive anatomical segments. Single mouse pointer clicks on these snapshots in the 3-D views were used to define target and entry points for the needle insertion plans (FIG. 16).

Targeting error in the accuracy tests was defined as distance of the needle tip from the surface of targeted copper spheres. Insertion time was defined as time from the definition of the insertion plan in the navigation software until the final placement of the needle. Success in facet joint needle placement was defined as the radiographic image of the needle tip being between the articular processes in the postero-anterior fluoroscopic view, and overlapping the articular processes in the lateral view.

Targeting error and insertion time were expressed as mean±standard deviation. The success rates of needle insertions were expressed as percentages. Linear regression was used to analyze trends in targeting error and procedure time with repeated needle insertions. Success rate between TUSS navigation and the freehand US-guided method was compared using a Chi-square test. Significance was defined as p<0.05 in all statistical tests.

### Results

System accuracy and the human anatomy feasibility tests were executed by three operators. Thirty needles were successfully positioned for accuracy testing. Targeting error was 1.03±0.48 mm. Maximum targeting error was 1.93 mm. Time from needle plan definition until final needle placement was 42.0±9.17 s. Maximum insertion time was 60 s. Targeting error did not change significantly as the number of needle insertions increased within operators (FIG. 17). Insertion time somewhat decreased with repeated insertions, but this trend was not statistically significant.

Facet joint needle placements in the synthetic human spine phantom were successful at first attempt in 29 insertions out of the total 30 insertions (96.7%) by three operators (10 facet joints each). In the case of the single missed facet joint, post-procedure analysis confirmed that the needle was placed at the planned position; however, the operator confused the facet joint with the gap between the vertebral lamina and the transverse process.

Cadaveric lamb facet joint needle placements were completed by all the five operators. TUSS guidance resulted in a success rate of 47 out of 50 cases (94%) as confirmed by post-insertion orthogonal fluoroscopic images. With freehand US-guided needle placement, success rate was 44% (22 of 50), which is significantly lower (p<0.001) compared to TUSS-guided insertions. Furthermore, the insertion time was significantly less (36.1±28.7 s) with TUSS guidance compared to freehand US guidance (47.9±34.2 s).

### Discussion

The results show that TUSS navigated facet joint needle insertion was significantly more accurate than freehand needle insertion in a patient-based synthetic human spine phantom and in a cadaveric lamb model. These results suggest that EM-tracked facet joint injections may be routinely performed without ionizing radiation imaging. Post insertion fluoroscopic analysis and registration with CT-based bone surface models revealed that all of the few missed needle placements were due to inaccurate US localization of the facet joint by the operators. This indicates the importance of training before the procedure is introduced in clinical practice. Identification of the facet joint by US is not a straightforward task even with a profound knowledge of the spinal anatomy. Operators in this study had no prior experience in US-guided facet joint injections and did not practice other forms of US-guided needle insertions on a daily basis.

Ultrasound guidance methods use landmarks on the images that can be identified with high confidence. Since US provides only a limited view of the underlying structures, the needle path is planned relative to the landmarks. Selection of the landmarks is not limited to one US slice. Landmark points (e.g., fiducials) in the 3D Slicer software can be placed, named, and highlighted in US slices of different orientations. These landmarks can be observed for needle navigation in different 3-D views of the virtual scene, as in the methods described herein. It is expected that these methods are applicable to a broad range of clinical procedures, in addition to the facet joint injections of this example, using anatomical landmarks. For example, for spinal nerve blocks, US guidance has an advantage over more frequently used imaging modalities. That is, US may directly visualize the target nerve, while conventionally used fluoroscopy does not show sufficient soft tissue contrast.

In conclusion, TUSS navigation allows for significantly better success rate and lower insertion time in facet joint injections by medical residents than freehand US needle guidance. Operators achieved good needle placement accuracy immediately as they started to use this guidance technique, which can be attributed to the intuitive user interface. This method may enable US guidance to be routinely used in facet joint injections, improving the safety and accessibility of treatment in patient populations with spine diseases.

In procedures such as the foregoing, use of a reference device in accordance with the described embodiments ensures that the electromagnetic field used for tracking is not distorted, therefore indicating that the needle guidance is accurate. Also, the reference device ensures that the ultrasound image and the tracked tools appear in the navigation computer display aligned with the point of view of the operator. This is essential to make the navigated intervention intuitive for the operator.

The contents of all references, pending patent applications, and published patents cited throughout this application are hereby expressly incorporated by reference.

### EQUIVALENTS

Those skilled in the art will recognize or be able to ascertain variants of the embodiments described herein. Such variants are within the scope of the invention and are covered by the appended claims.

1. A reference device for surgery, comprising:
a base portion, including;
a socket that accepts a tracking sensor in a pre-defined orientation;
one or more reference divots that accept at least a portion of a surgical intervention tool, the one or more reference divots being substantially transparent to one or more imaging modalities; and
a plurality of anatomical direction markers that provide alignment of the reference device with a patient's anatomy.

2. The reference device of claim 1, wherein the base portion interfaces with a patient's anatomy substantially non-invasively.

3. The reference device of claim 1, wherein the base portion interfaces with an object fixed to the patient's anatomy.

3. The reference device of claim 1, wherein the base portion interfaces with a surface in proximity to a surgical invention site.

4. The reference device of claim 1, wherein the socket accepts an electromagnetic tracking sensor that is used as a reference point in tracking at least one of position, orientation, and trajectory of the surgical intervention tool in three-dimensional space.

5. The reference device of claim 1, wherein locations of the one or more reference divots are selected with respect to the orientation of the tracking sensor.

6. A method of medical imaging; comprising:
disposing a reference device in a selected orientation with respect to an intervention space of a subject, the reference device providing anatomical orientation of tracked medical images within the intervention space;
using an ultrasound imaging system to obtain tracked medical images of the intervention space; and
using the anatomical orientation provided by the reference device to display the tracked medical images in the intervention space in a perspective that corresponds to an operator's perspective.

7. The method of claim 6, further comprising displaying one or more of position, orientation, and trajectory of a tracked intervention tool with respect to the tracked medical images in the intervention space.

8. The method of claim 6, further comprising verifying at least one of position, orientation, and trajectory of the tracked intervention tool with respect to the tracked medical images in the intervention space, by placing the tracked intervention tool at one or more locations on the reference device, wherein the locations are known with respect to the position of a sensor associated with the reference device.

9. The method of claim 6, wherein verifying further comprises providing an indication to the system when the tracked intervention tool is disposed at each of the one or more locations.

10. The method of claim 6, further comprising disposing an electromagnetic sensor in a known position and orientation with respect to the reference device.

11. The method of claim 6, wherein the medical imaging system is an ultrasound imaging system or a tomographic imaging system.

12. The method of claim 6, further comprising aligning a tracked medical image with a volumetric medical image.

13. The method of claim 6, wherein the tracked medical image is an ultrasound image.

14. The method of claim 6, further comprising displaying the tracked medical images substantially in real time.

15. Programmed media for use with a computer, comprising:
a computer program stored on non-transitory storage media compatible with the computer, the computer program containing instructions to direct the computer to perform the following steps:
obtain tracked medical images of an intervention space from a medical imaging system; and
use anatomical orientation provided by a tracked reference device to display the tracked medical images in the intervention space in a perspective that corresponds to an operator's perspective.
